(19) United States
(12) Patent Application Publication
(10) Pub. No.: US 2005/0181022 A1
(43) Pub. Date: Aug. 18, 2005

(54) Confectionary products including agent for controlling weight

(76) Inventors: David J. Cai, Darien, IL (US); Michael J. Greenberg, Northbrook, IL (US)

Correspondence Address: BELL, BOYD & LLOYD LLC, P. O. BOX 1135, CHICAGO, IL, 60690-1135, US

(21) Appl. No.: 10/966,787

(22) Filed: Oct. 15, 2004

Related U.S. Application Data
(60) Provisional application No. 60/517,704, filed on Nov. 5, 2003.
(60) Provisional application No. 60/517,925, filed on Nov. 5, 2003.
(60) Provisional application No. 60/518,011, filed on Nov. 5, 2003.
(60) Provisional application No. 60/518,009, filed on Nov. 5, 2003.
(60) Provisional application No. 60/518,000, filed on Nov. 5, 2003.
(60) Provisional application No. 60/518,010, filed on Nov. 5, 2003.

(51) Int. Cl. A61K 35/78; A61K 9/68

(52) U.S. Cl. 424/440; 424/760; 424/739; 424/734; 424/756

(57) ABSTRACT

A confectionary product for weight control including a therapeutically-effective amount of an agent and methods of use are provided.

### BACKGROUND OF THE INVENTION

The present invention relates generally to confectionary products. More specifically, the present invention relates to products for controlling or reducing weight.

Of course, one of the age-old concerns has been weight gain. Indeed, recently much attention has focused on the apparent increase in the percent of the U.S. population that is characterized as obese.

A number of products and methods have been proposed and developed to assist one in controlling weight. These products run the gamut from diets and exercise programs, to drugs and other compositions, both over-the-counter and prescription, to even surgery. Many of these products and methods, although effective, in certain instances have drawbacks. Moreover, most products and methods are not one hundred percent effective.

Accordingly, the need still exists for methods and compositions for controlling and losing weight.

### SUMMARY OF THE INVENTION

The present invention provides compositions and methods for controlling and/or losing weight. To this end, the methods and compositions can be used to prevent unwanted weight gain as well as to assist in weight loss.

Pursuant to the present invention, a confectionary product is provided that includes a therapeutically-effective amount of: a heating and cooling agent; indulgent flavors; softness; a cooling agent; hardness; or a heating agent. Methods and compositions of using the confectionary product to control weight are provided.

To this end, the present invention provides a confectionary product including a therapeutically-effective amount of: a heating and cooling agent; indulgent flavors; softness; a cooling agent; hardness; or a heating agent.

In an embodiment, the present invention provides a confectionary product wherein the heating agent is chosen from the group consisting of:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin; and
Capsicum oleoresin.

In an embodiment, the present invention provides a confectionary product wherein the cooling agent is chosen from the group consisting of:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.

In an embodiment, the present invention provides a weight control product comprising a confectionary product including a therapeutically-effective amount of a heating agent chosen from the group consisting of:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
Gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin; and
Capsicum oleoresin; and
a therapeutically-effective amount of a cooling agent chosen from the group consisting of:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.

In an embodiment, the present invention provides a method of controlling weight gain comprising the steps of consuming, on an as-needed basis, a confectionary product including a therapeutically-effective amount of a heating agent, a cooling agent or a combination of the two in order to reduce the appetite of the consumer.

In an embodiment, the present invention provides a method of controlling or reducing weight in an individual comprising the steps of providing a chewing gum product that includes a therapeutically-effective amount of a heating and cooling agent, the heating agent chosen from the group consisting of:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
Gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin;
Capsicum oleoresin;
the cooling agent chosen from the group consisting of:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.
wherein the heating and cooling agent reduces the appetite in the patient when the gum is chewed.

In an embodiment, the present invention provides a method of controlling weight gain comprising the steps of chewing, on an as-needed basis, a chewing gum product including a therapeutically-effective softness in order to reduce the appetite of the consumer.

In an embodiment, the present invention provides a method of controlling or reducing weight in an individual comprising the steps of providing to a consumer a chewing gum product that includes a therapeutically-effective softness.

In an embodiment, the present invention provides a confectionary product wherein the indulgent flavors are chosen from the group consisting of: chocolate and cream flavors.

In an embodiment, the present invention provides a weight control product comprising a confectionary product including a therapeutically-effective amount of indulgent flavors chosen from the group consisting of: chocolate and cream flavors.

In an embodiment, the present invention provides a method of controlling weight gain comprising the steps of consuming, on an as-needed basis, a confectionary product including a therapeutically-effective amount of indulgent flavors in order to reduce the appetite of the consumer.

In an embodiment, the present invention provides a method of controlling or reducing weight in an individual comprising the steps of providing a chewing gum product that includes a therapeutically-effective amount of indulgent flavors chosen from the group consisting of: chocolate and cream flavors.

In an embodiment, the present invention provides a method of controlling weight gain comprising the steps of chewing, on an as-needed basis, a chewing gum product including a therapeutically-effective hardness in order to reduce the appetite of the consumer.

In an embodiment, the present invention provides a method of controlling or reducing weight in an individual comprising the steps of providing to a consumer a chewing gum product that includes a therapeutically-effective hardness.

An advantage of the present invention is to provide products for controlling weight gain.

Another advantage of the present invention is to provide products for weight loss.

Still further, an advantage of the present invention is to provide methods for controlling weight gain.

Still another advantage of the present invention is to provide methods for reducing weight.

Furthermore, an advantage of the present invention is to provide products that will satiate the appetite of a consumer.

Additionally, an advantage of the present invention is to provide products for controlling weight or for weight loss that can be dispensed over the counter and do not create any unreasonable health risk.

Additional features and advantages of the present invention are described in, and will be apparent from, the following Detailed Description of the Invention.

### DETAILED DESCRIPTION OF THE INVENTION

The present invention relates to confectionary products as well as methods of using same for controlling weight or reducing weight in a consumer. Pursuant to the present invention, a confectionary product is provided that includes: a heating and/or cooling agent; indulgent flavors; softness; or hardness.

These components can be used in a variety of confectionary products. In a preferred embodiment, the confectionary product is chewing gum. However, other confectionary products can be utilized such as, for example, hard candy, soft candies, boiled candies, and edible film strips.

Pursuant to the present invention, a therapeutically-effective amount of heating and/or cooling agent can be provided. “Therapeutically-effective amount” means a sufficient amount of agent to reduce or satiate the appetite of an individual consuming the product.

As used herein, the term “heating” and “cooling agent” refers to an agent that creates a sensation in the buccal cavity or mouth of the consumer such as a heating and cooling (depending on the agent), tingling, or itching sensation. A variety of heating and cooling agents can be utilized. By way of example, and not limitation, the following heating agents can be utilized either alone or in combination:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
Gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin; and
Capsicum oleoresin.

By way of example and not limitation, the following cooling agents can be utilized:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.

Generally, the heating and cooling agent, in combination, should be present in an amount of from about 0.01% to about 5% and preferably 0.01% to 2% by weight. Of course, certain heating and cooling agents may be used in greater or lesser amounts due to the effect to be achieved. Likewise, depending on the type of confectionary product, a greater or lesser amount of heating and cooling agent may be desired. In an embodiment, only a minor amount of heating agent is used.

By way of example and not limitation, capsicum oleoresin, capsaicin, piperine, gingerol, and shoagol are in an embodiment used in quantities from about 0.01% to about 0.03%. Cinnamic aldehyde can be used in amounts from about 1% to 2%. The oleoresins can be used in amounts from 0.05% or less. All of the above is by weight and all would be with a cooling agent.

Of course, the amounts can also vary depending on whether a single or a combination of heating and cooling agents are used.

Although not wishing to be bound by any theory, it is believed that the heating and/or cooling agent in the confectionary will stimulate the trigeminal nerve. By so stimulating the trigeminal nerve, it is believed this will reduce the consumer's appetite. In part, this is achieved, it is believed, because trigeminal stimulation increases circulating norepinepherin. This can cause an increase in alertness and blood flow to the brain; a flight or fight situation. This, in turn, will reduce or eliminate the appetite.

Furthermore, due to the heating and/or cooling agent, saliva flow in the mouth can be increased. This likewise has the effect of, the inventors believe, satiating the appetite. In a preferred embodiment, the heating and cooling agent, or a combination thereof, is incorporated in a chewing gum product.

Pursuant to the present invention, a chewing gum product can be provided that includes a sufficient softness. As used herein, the term “softness” refers to a state that is softer, less rigid, than traditional chewing gum products. A variety of ranges of softness can be utilized.

Thus, the present invention can provide a chewing gum having a therapeutically-effective softness. “Therapeutically-effective softness” means a softness that is sufficient to reduce or satiate the appetite of an individual consuming the product. This softness is less than that of traditional chewing gum. In this regard, by way of example and not limitation, the gum can have between 90% to 25% of the firmness of traditional gum, and preferably 80-50% of the firmness. There are a variety of ways to measure the softness of chewing gum including using an Ingstrom device.

Although not wishing to be bound by any theory, it is believed that the softness of the chewing gum will cause the consumer to chew or masticate more. The soft texture promotes mastication which reduces appetite and/or food intake. It should be noted that a lack of chewing contributes partly to high food intake in overweight individuals. Mastication certainly plays a role in controlling eating behavior.

Furthermore, the soft texture can cause the saliva flow in the mouth of the consumer to increase. This likewise has the effect of, the inventors believe, satiating the appetite.

Pursuant to the present invention, a chewing gum product can be provided that includes a sufficient hardness. As used herein, the term “hardness” refers to a state in which the chewing gum product provides more resistance to chewing in the chewing process. A variety of ranges of hardness can be utilized.

Thus, the present invention can provide a chewing gum having a therapeutically-effective hardness. “Therapeutically-effective hardness” means a hardness, or firmness, that is sufficient to reduce or satiate the appetite of an individual consuming the product. This hardness or rigidity is greater than that of traditional chewing gum. In this regard, by way of example and not limitation, the gum can be between 125% to 500% harder than traditional gum, and preferably 150-300% harder. There are a variety of ways to measure the hardness of chewing gum including using an Ingstrom device.

Although not wishing to be bound by any theory, it is believed that the hardness of the chewing gum will cause a signal to be sent to the brain indicating that aggressive chewing is ongoing, and that therefore any signals to the brain indicating a need for caloric intake, energy intake, or general hunger, can be ignored, as the body believes such is being chewed already to satisfy this demand.

Furthermore, the hard texture can cause the saliva flow in the mouth of the consumer to increase. This likewise has the effect of, the inventors believe, satiating the appetite.

Pursuant to the present invention, a confectionary product can be provided that includes an indulgent flavor. As used herein, the term “indulgent flavors” refers to an agent that creates a sensory satisfaction and thereby satisfies a craving for the flavor rather than carbohydrates. A variety of indulgent flavors can be utilized. By way of example, and not limitation, the following indulgent flavors can be utilized either alone or in combination: chocolate and cream flavors.

Indulgent flavors can be used in a variety of confectionary products. In a preferred embodiment, the confectionary product is chewing gum. However, other confectionary products can be utilized such as, for example, hard candy, soft candies, boiled candies, and edible film strips.

A therapeutically-effective amount of indulgent flavors can be provided. “Therapeutically-effective amount” means a sufficient amount of flavors to reduce or satiate the appetite of an individual consuming the product. Generally, the indulgent flavors should be present in an amount of from about 0.01% to about 20% by weight and preferably, 0.2% to 0% by weight. Of course, certain indulgent flavors may be used in greater or lesser amounts due to the effect to be achieved. Likewise, depending on the type of confectionary product, a greater or lesser amount of indulgent flavors may be desired. By way of example, a confectionary product can be provided including 1% to 2% by weight chocolate flavor. The amounts can also vary depending on whether a single or a combination of indulgent flavors are used.

Although not wishing to be bound by any theory, it is believed that the indulgent flavors in the confectionary satisfies the consumer's craving for these flavors. By satisfying this craving, it is believed this will reduce the consumer's appetite and/or intake of carbohydrates. To this end, it is believed the individual craves the flavor, not necessarily the carbohydrates.

Furthermore, certain indulgent flavors can cause the saliva flow in the mouth to increase. This likewise has the effect of, the inventors believe, satiating the appetite. In a preferred embodiment, the indulgent flavors, or a combination thereof, is incorporated in a chewing gum product.

In any of the above embodiments, the chewing gum product of the present invention can be any chewing gum product known in the art.

A variety of different chewing gums and formulations can be utilized. Such chewing gums include a water insoluble gum base, a water soluble portion, and flavor. The water soluble portion dissipates with a portion of the flavor of the gum over a period of time during chewing. The gum base portion is retained in the mouth throughout the chew.

The insoluble gum base generally comprises elastomers, resins, fats and oils, softeners and inorganic fillers. The gum base may or may not include wax. Typically, gum base comprises approximately 20% to about 40% of the gum product. The insoluble gum base can constitute approximately 30% to about 90% by weight of the chewing gum, in an embodiment, the gum base comprises at least 50% of the chewing gum.

In an embodiment, the chewing gum base of the present invention contains about 20% to about 60% by weight synthetic elastomer, about 0% to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers may include, but are not limited to, polyisobutylene with GPC weight average molecular weight of about 10,000 to about 95,000, isobutylene-isoprene copolymer (butyl elastomer), styrene-butadiene, copolymers having styrene-butadiene ratios of about 1:3 to about 3:1, polyvinyl acetate having GPC weight average molecular weight of about 2,000 to about 90,000, polyisoprene, polyethylene, vinyl acetate—vinyl laurate copolymer having vinyl laurate content of about 5% to about 50% by weight of the copolymer, and combinations thereof.

Preferred ranges for polyisobutylene are 50,000 to 80,000 GPC weight average molecular weight and for styrene-butadiene are 1:1 to 1:3 bound styrene-butadiene, for polyvinyl acetate are 10,000 to 65,000 GBC weight average molecular weight with the higher molecular weight polyvinyl acetates typically used in bubble gum base, and for vinyl acetate-vinyl laurate, vinyl laurate content of 10-45%.

Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum, as discussed below. Preferred natural elastomers include jelutong, chicle, sorva and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha-pinene, beta-pinene, and/or d-limonene; and any suitable combinations of the foregoing. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-calcium phosphate, cellulose polymers, such as wood, and combinations thereof.

Softeners/emulsifiers may include tallow, hydrogenated tallow, hydrogenated and partially hydrogenated vegetable oils, cocoa butter, glycerol monostearate, glycerol triacetate, lecithin, mono-, di- and triglycerides, acetylated monoglycerides, fatty acids (e.g. stearic, palmitic, oleic and linoleic acids), and combinations thereof.

Colorants and whiteners may include FD&C-type dyes and lakes, fruit and vegetable extracts, titanium dioxide, and combinations thereof.

The base may or may not include wax. An example of a wax-free gum base is disclosed in U.S. Pat. No. 5,286,500, the disclosure of which is incorporated herein by reference.

In addition to a water insoluble gum base portion, a typical chewing gum composition includes a water soluble bulk portion and one or more flavoring agents. The water soluble portion can include bulk sweeteners, high-intensity sweeteners, flavoring agents, softeners, emulsifiers, colors, acidulants, fillers, antioxidants, and other components that provide desired attributes.

Softeners are added to the chewing gum in order to optimize the chewability and mouth feel of the gum. The softeners, which are also known as plasticizers and plasticizing agents, generally constitute between approximately 0.5% to about 15% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide-containing components commonly known in the chewing gum art, including but not limited to, sucrose, dextrose, maltose, dextrin, dried invert sugar, fructose, levulose, glactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High-intensity artificial sweeteners can also be used, alone or in combination, with the above. Preferred sweeteners include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent can be used. Examples of low caloric bulking agents include: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion.

In use, the chewing gum product, or other confectionary product, would be chewed by the consumer on an as-needed basis. This could be prophylactically during the day to discourage snacking and control the desire to eat. Likewise, the chewing gum can be chewed immediately before a meal to reduce the caloric intake during the meal or after the meal to discourage the desire for dessert. A variety of maintenance plans and regimens for either reducing one's weight or controlling one's weight can be imagined.

Likewise, this product can be used in conjunction with other products or other methods. For example, an obese individual can be placed on an exercise program as well as a diet that reduces caloric intake or the intake of certain foods, e.g., the Atkins Diet. At the same time, a consumer could utilize the confectionary product of the present invention to assist them in maintaining and/or following the diet. Of course, if desired, the products of the present invention can be used alone to either or control or reduce weight.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A confectionary product including a therapeutically-effective amount of a component selected from the group consisting of: a heating agent; a cooling agent; an indulgent flavor; softness; hardness; and combinations thereof.

2. The confectionary product of claim 1 wherein the heating agent is chosen from the group consisting of:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
Gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin; and
Capsicum oleoresin.

3. The confectionary product of claim 1 wherein the cooling agent is chosen from the group consisting of:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.

4. A weight control product comprising a confectionary product including a therapeutically-effective amount of a heating agent chosen from the group consisting of:
Cinnamic aldehyde;
Vanillyl Butyl Ether;
Capsicum (Red Pepper) and its derivatives;
Cayenne (Capsicum annuum L);
Paprika and its extract (capsorubin, capsanthin);
Trans-8-Methyl-n-Vanillyl-6-nonenamide Capsaicin;
Piperine (Black Pepper);
Wasabi;
Ginger root and its extract;
Gingerol;
Sanshools;
Szechwan (Sichuan) Pepper and its extract;
Shoagol;
Ginger oleoresin;
Cassia oleoresin;
Black pepper oleoresin;
Capsaicin;
Cinnamon;
Pepper oleoresin;
Capsicum oleoresin; and
a therapeutically-effective amount of a cooling agent chosen from the group consisting of:
WS-3;
WS-23;
WS-30;
WS-14;
Eucalyptus extract (p-Mehtha-3,8-Diol);
Menthol (its natural or synthetic derivatives);
3,3,5-Trimethyl Cyclohexanol;
Ethyl p-menthane carboxamide;
N,2,3-trimethyl-2-isopropyl butanamide;
Menthyl glutarate FEMA 4006;
Menthyl succinate;
Menthol PG carbonate;
Menthol EG carbonate;
Menthyl lactate;
Menthone glyceryl ketal;
Menthol glyceryl ether;
N-tertbutyl-p-menthane-3-carboxamide;
P-menthane-3-carboxylic acid glycerol ester;
Methyl-2-isopryl-bicyclo (2.2.1);
Heptane-2-carboxamide; and
Menthol methyl ether and combinations thereof.

5. A method of controlling weight gain comprising the steps of consuming, on an as-needed basis, a confectionary product including a therapeutically-effective amount of a component selected from the group consisting of a heating agent; a cooling agent; an indulgent flavor; softness; and hardness in order to reduce the appetite of the consumer.

6. A weight control product comprising a chewing gum including a therapeutically-effective hardness that is at least 125% firmer than traditional chewing gum.

7. A confectionary product including a therapeutically-effective amount of indulgent flavors.

8. The confectionary product of claim 1 wherein the indulgent flavors are chosen from the group consisting of: chocolate and cream flavors.

9. A weight control product comprising a chewing gum including a therapeutically-effective softness that is no more than 90% of the firmness of traditional chewing gum.
